(19) United States
(12) Patent Application Publication
(10) Pub. No.: US 2006/0024391 A1
(43) Pub. Date: Feb. 2, 2006

(54) Dietary nutritional suppliments for persons smoking tobacco products

(76) Inventor: Zarahmehran Salari Lak, Gibsonia, PA (US)

Correspondence Address: BLYNN L. SHIDELER;THE BLK LAW GROUP, 3500 BROKKTREE ROAD, SUITE 200, WEXFORD, PA, 15090, US

(21) Appl. No.: 11/196,847

(22) Filed: Aug. 1, 2005

Related U.S. Application Data
(60) Provisional application No. 60/592,584, filed on Jul. 30, 2004.

(51) Int. Cl. A61K 36/236; A61K 36/16; A61K 38/43; A61K 33/32; A61K 33/04

(52) U.S. Cl. 424/728; 424/773; 424/752; 424/641; 424/764; 424/94.1; 514/52; 514/356; 514/474; 424/702

(57) ABSTRACT

A dietary nutritional supplement is designed specifically to address the needs of tobacco users, in particular smokers. The nutritional supplement for smokers contains effective amounts of vitamin B complex, vitamin B₁₂ (Cobalamin), Vitamin B₃ (Niacin), vitamin C (Ascorbic acid), L-cysteine, L-methionine, co-enzyme Q₁₀, milk thistle, ginko, ginseng, selenium, yerb santa, marshmallow root, and zinc.

### CROSS REFERENCE TO RELATED APPLICATION

The present application claims the benefit of U.S. provisional patent application Ser. No. 60/592,484 entitled “NUTRITIONAL SUPPLEMENTS FOR PERSONS SMOKING TOBACCO PRODUCTS” filed on Jul. 30, 2004.

### BACKGROUND OF THE INVENTION

1. Field of the Invention

The present invention relates to a dietary nutritional supplements for humans, and more particularly to dietary nutritional supplements for humans who consume tobacco products (i.e. smokers).

2. Background Information

Tobacco has been used as a mood-altering substance for centuries. It has been chewed, sniffed, and smoked, and is now most commonly used in the form of cigarettes. Tobacco comes from the plant nicotiana tabacum. Tobacco is used by so many because it contains nicotine. Further, the ‘habit’ itself is also addictive, most people who smoke DO like it. Cigarette smoke contains thousands of chemicals; many are responsible for its powerful addictiveness. When tobacco is smoked, nicotine is absorbed by the lungs and quickly transported into the bloodstream, where it is circulated through the brain. This all happens very rapidly, so fast in fact that, nicotine reaches the brain within 8 seconds after someone inhales tobacco smoke. Nicotine can also enter the bloodstream through the mucous membranes that line the mouth (if tobacco is chewed) or nose (if snuff is used), and even through the skin. Nicotine acts as a stimulant on the central nervous system and also has many metabolic affects. It increases the metabolic rate, raises blood pressure, changes muscle tension and affects certain brain chemicals and hormones.

Smoking has a detrimental effect on nutrition. Smokers break down vitamin C twice as fast as nonsmokers. Vitamin C is believed to be one of the most powerful antioxidants for humans. Other vitamins and nutrients that the body needs are also depleted by smoking. Inhaling cigarette smoke leads to an increased amount of free radicals in the body. Defined simply, a free radical is an atom or group of atoms that can cause damage to the body. Free radicals are normally present in the body in small numbers. In large amounts they can cause damage to body cells and tissues. Under normal conditions, anti-oxidants protect the body against free radical damage. An anti-oxidant defends against damage to the body by binding to (joining with) the free radical. This keeps other bodily substances from being altered or destroyed.

Vitamins A, C, E, and Beta-carotene are all anti-oxidants. Due to the increased amount of free radical activity, smoking depletes a human body's supply of these anti-oxidants very quickly. Remaining free radicals, that are allowed to go unmonitored, slowly cause damage that can result in heart disease or various types of cancer.

It is for these reasons that a smoker, or someone who is routinely exposed to second hand smoke, must obtain an adequate supply of anti-oxidants through their diet or dietary supplements. The RDA (Recommended Dietary Allowances) for Vitamin C for non-smokers is 60 mg/day. For a smoker it increases to 100 mg/day.

As reported in Vitamin and Nutrition Research (March 2000) a prospective survey was conducted in a random sample of smokers (SMO) and non-smokers (NON) men. The aim of the survey was to investigate metabolic and dietary parameters discriminating between SMO and NON volunteers. Forty three blood markers were determined and dietary intakes evaluated during four seasonal periods. Alpha-carotene, beta-carotene, beta-cryptoxanthin, total carotenoids and riboflavin blood concentrations were lower in SMO vs. NON subjects. Activities of glutathione reductase and butryrylcholinesterase were lower in the SMO group, as did total protein levels. Copper and thiocyanates were higher in SMO subjects. Most of the dietary parameters did not differ between the two groups of volunteers. Low biologically available quantities of carotenoids and riboflavin from low dietary intakes and depletion through smoking would be the main precipitating factors of Epidemic Neuropathy. Protein depletion and copper delivery via tobacco smoke, sunlight exposure, level of physical activity and high consumption of cigarettes, in a context of low dietary supplies concerning especially B vitamins, were probably aggravating factors through oxidative stress and impairment of cyanide detoxification.

There are many studies evidencing the nutritional depletion of the body due to smoking. It is an object of the present invention to provide a dietary nutritional supplement that is designed specifically to address the needs of tobacco users, in particular smokers.

### SUMMARY OF THE INVENTION

The above stated objects achieved with a dietary nutritional supplement according to the present invention. The nutritional supplement according to the present invention contains effective amounts of vitamin B complex, vitamin B₁₂ (Cobalamin), Vitamin B₃ (Niacin), vitamin C (Ascorbic acid), L-cysteine, L-methionine, co-enzyme Q₁₀, milk thistle, ginko, ginseng, selenium, yerb santa, marshmallow root, and zinc. The dietary supplement according to the present invention is designed to address the vitamin depletion experienced by users of tobacco products, cleanse the body of toxins associated with tobacco products, improve the functioning of the body and the healing of the body, and prepare the body for the withdrawal of tobacco products (which will assist the user in terminating the use of tobacco products).

The above supplement may be in an liquid carrier, such as a fruit juice (e.g. apple juice or beet juice) or dairy based (e.g. milk or yogurt). The supplement of the present invention may be formed in a baked good or confection such as a biscuit, cookie, chocolate bar (also known as energy bars). The supplement of the present invention may be in pill or tablet form. The supplement may also be in powder form to be added to a carrier liquid, such as water. Other forms of the supplement are also possible.

These and other advantages of the present invention will be clarified in the description of the preferred embodiment.

### DESCRIPTION OF THE PREFERRED EMBODIMENTS

The nutritional supplement according to the preferred embodiment of the present invention contains effective amounts of vitamin B complex, vitamin B₁₂ (Cobalamin), Vitamin B₃ (Niacin), vitamin C (Ascorbic acid), L-cysteine, L-methionine, co-enzyme Q₁₀, ginko, milk thistle, ginseng, selenium, yerb santa, marshmallow root, and zinc.

Yerba Santa, Eriodictyon californisum, is an herb with known medicinal properties. Yerba Santa is an evergreen shrub native to the western United States. Yerba Santa has been used as an expectorant to treat coughs and congestion, as well as aiding in loosening and expelling phlegm. It works to dilate bronchial tubes. It has been used to ease asthma and allergy attacks. A poultice of the mashed leaves of the herb has also been used for sores and wounds. In a single daily dosage of the supplement according to the present invention Yerba Santa is used in an effective amount of 2-8 CC, most preferably 5 CC. Commercial forms of the herb (extract) may be in liquid form.

Coenzyme Q₁₀ (also known as CoQ₁₀, Q₁₀, vitamin Q₁₀, ubiquinone, or ubidecarenone) is a compound that is made naturally in the body. Coenzyme Q₁₀ is used by cells to produce energy needed for cell growth and maintenance and is also used by the body as an antioxidant. Coenzyme Q₁₀ has been used as a dietary supplement in patients undergoing cancer treatment. In these studies, the researchers explored the potential use of coenzyme Q₁₀ as an adjuvant therapy for cancer. There have also been anecdotal reports that coenzyme Q₁₀ has increased the survival of patients with cancers of the pancreas, lung, colon, rectum, and prostate. In a single daily dosage of the supplement according to the present invention co enzyme Q₁₀ is used in an effective amount of 100-300 Mgs, most preferably 200 Mgs.

Milk Thistle, Silybum marianum, is an herb that has been used medicinally for functional disorders of the liver and gallbladder. It has been considered especially helpful in cases jaundice, colitis, pleurisy, and diseases of the spleen. Milk thistle is indigenous to the South of Europe however today it can be found growing around the world. It is naturalized in most of Europe and North America. Milk Thistle's main active bio-constituent is Silymarin. Silymarin selectively acts as an anti-oxidant and protects the body from free radical damage specifically in the intestines and stomach. It increases the liver's content of GSH (glutathione) which is a substance used by the body in detoxifying many potentially damaging hormones, chemicals, and drugs (including acetaminophen) It has demonstrated a membrane stabilizing action, which inhibits or prevents lipid peroxidation. It seems to alter the structures of outer wall membranes of hepatocytes, preventing penetration of liver poisons and stimulates the action of nuclear polymerase. It may increase ribosomal protein synthesis and stimulate the formation of new hepatocytes. Milk Thistle in the preferred embodiment of the present invention is used in an effective amount of ½-1½ tablespoons (about 7-20 ml), most preferably 1 tablespoon (about 15 ml). The commercial version of Milk Thistle is in powder form.

An optional additive not found in the most preferred embodiment of the present invention, Liorice root, Glycyrrhiza glabra, is also known by the names Liquorice, Yashtimadhu, The Great Harmonizer, Grandfather Herb, and Sweetwood. Licorice is a perennial herb native to southern Europe, Asia and the Mediterranean. It is also extensively cultivated in Russia, Spain, Iran and India.

Historically licorice has been known to contain many health benefits. Ancient cultures on every continent have used Licorice Root, the first recorded use by the Egyptians in the 3rd century BC. The Egyptians and the Greeks recognized the herb's benefits in treating coughs and lung disease. Licorice is the second most prescribed herb in China, where it is suggested for treatment of the spleen, liver and kidney. The Japanese use a Licorice preparation to treat hepatitis. At one time in Germany, Licorice was a more popular gift to give than chocolate as it was reputed to sexually arouse women. The most common medical use for Licorice Root is for treating upper respiratory ailments including coughs, hoarseness, sore throat, and bronchitis.

The main constituent found in the root is glycyrrhizin. The plant also contains various sugars (up to 14%), starches (30%), flavonoids, saponoids, sterols, amino acids, gums, and essential oil. Glycyrrhizin stimulates the secretion of the adrenal cortex hormone “aldosterone”. This herb can be as effective as codeine, and safer, when used as a cough suppressant. Rhizomes in Licorice have a high mucilage content which, when mixed with water or used in cough drops, sooths irritated mucous membranes. The use of Licorice also has an expectorant effect which increases the secretion of the bronchial glands. Today, herbal preparations containing Licorice Root are used to treat stomach and intestinal ulcers, lower acid levels and coat the stomach wall with a protective gel. Rarely used alone, Licorice is a common component of many herbal teas as a mild laxative, a diuretic, and for flatulence. It has also been known to relieve rheumatism and arthritis, regulate low blood sugar, and is effective for Addison's disease. The Root extract produces mild estrogenic effects, and it has proven useful in treating symptoms of menopause, regulating menstruation, and relieving menstrual cramps. It is also widely used in other medicines to mask bitter tastes and also to prevent pills from sticking together. Licorice has also been used in poultices for treatment of dermatitis and skin infections. It helps to open the pores and is used in combination with other cleansing and healing herbs as an emollient. The common name Licorice also includes the species Glycyrrhiza uralensis, which is used interchangeably with Glycyrrhiza glabra. Licorice is not found in the supplement according to the preferred embodiment of the present invention, but Licorice may be used as an optional additive in an alternative to the preferred embodiment of the present invention and may be used as an alternative additive in an effective amount of 1-3 grams, most preferably 2 grams.

Marshmallow root, Althaea officinalis, is also known by the names Althea, Mallards, Sweet Weed, Hock Herb, Wymote, and Schloss Tea. The genus name Althea is from the Greek word “althe” and means “to heal”. Marshmallow Root powder has been used as a binding agent to hold other herbs together in making pills, and has been commonly substituted for Slippery Elm in herbal remedies. Marshmallow is a native of most countries of Europe, from Denmark southward, and is found in the western U.S.

Primary chemical constituents of Marshmallow include mucilage, polysaccharides, flavonoids (quercetin, kaaempferol), asparagine, tannins, lecithin, and pectin. The great demulcent and emollient properties of Marshmallow make it useful in inflammation and irritation of the alimentary canal, and of the urinary and respiratory organs. Marshmallow has also been used as an expectorant to treat a variety of upper respiratory problems. Marshmallow contains large amounts of vitamin A, calcium, zinc and significant amounts of iron, sodium, iodine, and B-complex. Like slippery elm, Marshmallow reduces inflammation and has a calming effect on the body. The active constituents in Marshmallow are large carbohydrate (sugar) molecules, which make up the mucilage. This smooth, slippery substance can soothe and protect irritated mucous membranes. Although Marshmallow has primarily been used for the respiratory and digestive tracts, its high mucilage content may also provide some minor relief for urinary tract and skin infections. Marshmallow's mucilage content helps soothe inflamed tissues, often caused by bronchitis and asthma. Marshmallow also relieves dryness and irritation in the chest and throat, usually brought on by colds and persistent coughs. Marshmallow has been known to relieve indigestion, kidney problems, urinary tract infections, and even external skin wounds such as boils and abscesses. In a single daily dosage of the supplement according to the preferred embodiment of the present invention Marshmallow is used in an effective amount of 2-8 ml, most preferably 5 ml. The commercially available form of marshmallow root is in powder form.

An optional additive not found in the most preferred embodiment of the present invention, Goldenseal, Hydrastis canadenis, is a native American medicinal plant introduced to early settlers by Cherokee Indians who used it as a wash for skin diseases, wounds, and for sore, inflamed eyes. Its roots are bright yellow, thus the name. Goldenseal root has acquired a considerable reputation as a natural antibiotic and as a remedy for various gastric and genitourinary disorders.

Numerous references to Goldenseal began to appear in medical writings as far back as 1820 as a strong tea for indigestion. Today it is used to treat symptoms of the cold and flu and as an astringent, antibacterial remedy for the mucous membranes of the body. The active ingredients in Goldenseal are the alkaloids hydrastine and berberine. Similar in action, they destroy many types of bacterial and viral infections. These alkaloids can also reduce gastric inflammation and relieve congestion. Berberine is a bitter that aids digestion and that has a sedative action on the central nervous system. Although not in the preferred embodiment of the present invention, Goldenseal may be used as an optional additive in an alternative embodiment according to the present invention and may be used in an effective amount of 250-750 mg, most preferably 500 mg.

An optional additive not found in the most preferred embodiment of the present invention, Maitake, Grifola frondosa, is the Japanese name for an edible fungi (i.e. mushroom) with a large fruiting body characterized by overlapping waves. Many refer to Maitake as the “dancing mushroom”. Maitake is cultivated as a food and as a dietary supplement that is believed to significantly benefit overall health. Among the most important constituents, however, are certain polysaccharides known as the beta-glucans. Maitake's prominent immune-boosting effects are thought to be due predominantly to these polysaccharides. These “biological response modifiers” are thought to be potent anti-viral and anti-tumor agents, not by killing viruses or cancer cells directly but by stimulating the body's innate ability to marshal cellular defenses. The maitake mushroom contains a high percentage of beta glucan. Although not in the preferred embodiment of the present invention, Maitake may be used as an optional additive in an alternative to the preferred embodiment in an effective amount of 500-1500 mg, most preferably 1000 mg.

L-Cysteine is a conditionally essential amino acid and antioxidant. L-Cysteine is one of only three sulfur-containing amino acids, the others being taurine (which can be produced from L-cysteine) and L-methionine from which L-cysteine can be produced in the body by a multi-step process. L-cysteine can act as an antioxidant, may prevent liver diseases, and can help to thicken the individual diameters of existing hair if taken regularly. In a single daily dosage of the supplement according to the present invention L-Cysteine is used in an effective amount of 250-750 mg, most preferably 500 mg.

L-Methionine is an essential sulfur amino acid that aids in the body's detoxification processes. Sunflower seeds are a great source of Methionine. Methionine has been used to treat depression, alcoholism, liver disease, and reduce allergies and asthma. It has been used to reduce the toxic effects of radiation. This amino acid improves healing from surgery and other wounds. Some studies suggest that Methionine may reduce withdrawal symptoms and depression from heroin, barbiturates, or amphetamines. It can also be used as an adjunct to L-dopa treatment for Parkinson's disease. In a single daily dosage of the supplement according to the present invention L-Methionine is used in an effective amount of 250-750 mg, most preferably 500 mg.

An optional additive not found in the most preferred embodiment of the present invention, Vitamin E, tocopherol, is a major anti-oxidant nutrient; retards cellular aging due to oxidation; supplies oxygen to the blood which is then carried to the heart and other organs; thus alleviating fatigue; aids in bringing nourishment to cells; strengthens the capillary walls prevents the red blood cells from destructive poisons; prevents dissolves blood clots; has also been used by doctors in helping prevent sterility, muscular dystrophy, calcium deposits in blood walls and heart conditions. Although not found in the preferred embodiment of the present invention, Vitamin E may be used as an optional additive in an alternative to the preferred embodiment in an effective amount of 100-300 IU, most preferably 200 IU.

Vitamin C, ascorbic acid or ascorbate, is an antioxidant vitamin that protects cells from oxidative damage. Vitamin C is necessary for the production of collagen (e.g., for wound healing), hormones and neurotransmitters; it may have a role in fighting infection. Deficiency results in poor healing, easy bruising and anemia. Vitamin C is a water-soluble vitamin found in plants, especially in fruits and in leafy vegetables, or made synthetically and helps wounds heal, strengthens blood vessels, builds connective tissue, healthy gums, skin and promotes strong teeth and bones, and may boost immunity. In a single daily dosage of the supplement according to the present invention Vitamin C is used in an effective amount of 1000-3000 Mg, most preferably 2000 Mg.

An optional additive not found in the most preferred embodiment of the present invention, Folic acid is a B complex water-soluble vitamin. It functions as a coenzyme in the breakdown and utilization of protein, together with vitamins B12 and C. Folic acid functions as a carbon carrier in the formation of heme, the iron-containing protein found in hemoglobin, necessary for the formation of red blood cells. In addition, folic acid is needed for the formation of nucleic acid which is necessary for the growth and reproduction of body cells. Disease prevention therapy with sulfa-containing drugs may interfere with the bacteria in the intestinal tract which are responsible for manufacturing folic acid. As a result, a deficiency of this vitamin may occur. Folic acid is known to decrease the occurrence of some birth defects such as open neural tube defects. Although not found in the preferred embodiment of the present invention, Folic Acid may be used as an additive in alternative embodiment in an effective amount of 200-600 Mcg, most preferably 400 Mcg.

Vitamin B12, Also known as cyanocobalamin, is a water-soluble vitamin necessary for normal metabolism of nerve tissues and is active in carbohydrate, fat, and protein metabolism. B12 improves the functions of iron in the metabolic cycle and assists folic acid in the synthesis of choline. It also plays an important role in the production of DNA and RNA, the genetic material of living cells. Vitamin B12 helps in the formation regeneration of red blood cells, thus helping prevent anemia; necessary for carbohydrate, fat protein metabolism; maintains a healthy nervous system; promotes growth in children; increases energy; needed for Calcium absorption. Low vitamin B12 levels damage nerve cells, aggravate nervous system disorders, and cause anemia. The prevalence of vitamin B12 deficiency increases with age, especially over 65, and is frequently associated with Alzheimer's disease. Vitamin B12 deficiency is also reported to be a risk factor for heart disease, stroke and accelerated aging. In a single daily dosage of the supplement according to the present invention Vitamin B12 used in an effective amount of 500-1500 Mcg (0.5-1.5 Mg), most preferably 1000 Mcg (1 Mg).

Vitamin B complex is actually a combination of eleven different vitamins, namely: thiamin (B1), riboflavin (B2), niacin (B3), folic acid, biotin, pantothenic acid or panthenol (B5), pyridoxine (B6), choline, inositol, PABA, and cyanocobalamin (B12). Collectively the Vitamin B Complex are antioxidants and body regulators that work better together than alone. In a single daily dosage of the supplement according to the present invention Vitamin B complex used in an effective amount of 50-150 Mg, most preferably 100 Mg. The vitamin B complex in the present formulation is in addition to the vitamin B 12 discussed above and B3 (even though B complex will include B12 and B3). As known to those of ordinary skill in the art, B12, B3 and B complex are separately commercially available.

Ginkgo, Ginko Biloba, comes from the leaves ginkgo plant with most commercial leaf production from plantations in South Carolina, France, and China. The leaves were traditionally used for “benefiting the brain”, treatment of lung disorders, relief of cough and asthma symptoms, and diarrhea. The leaf tea was applied externally to treat sores of the skin and remove freckles. Complex extracts have been used to increase circulation to the extremities as well as the brain, especially in the elderly. Ginkgo extract has also been studied for the treatment of ringing in the ears (tinnitus), male impotence, degenerative nerve conditions such as multiple sclerosis, and other diseases. It has shown potential to relieve difficulties with short-term memory, attention span, and mood in early stages of Alzheimer's disease by improving oxygen metabolism in the brain. Ginkgo's effects have been attributed to compounds called flavone glycosides, as well as unique compounds—ginkgolides—which are potent inhibitors of a platelet-activating factor involved in the development of inflammatory, cardiovascular, and respiratory disorders. The ginkgolides' activity helps explain the herb's broad-spectrum biological effects. Another important effect is strong antioxidant activity. With its ability to “scavenge” reactive oxygen forms known as free radicals, ginkgo leaf extract directs antioxidant effects to the brain, central nervous system, and cardiovascular system. This is one of the mechanisms that make it promising in treatment of age-related declines of brain function. In a single daily dosage of the supplement according to the present invention Ginko is used in an effective amount of 5-15 cc, most preferably 10 cc.

Ginseng, Panax ginseng, is the most famous Chinese herb. It is the most widely recognized plant used in traditional medicine. Various forms of ginseng have been used in medicine for more than 7000 years. Several species grow around the world, and though some are preferred for specific benefits, all are considered to have similar properties as an effective general rejuvenator. The name panax is derived from the Greek word panacea meaning, “all healing” and the benefits of ginseng are recognized as such. Ginseng is commonly used as an adaptogen, meaning it normalizes physical functioning depending on what the individual needs (for example, it will lower high blood pressure, but raise low blood pressure). It is also used to reduce the effects of stress, improve performance, boost energy levels, enhance memory, and stimulate the immune system. Ginseng contains vitamins A, B-6 and the mineral Zinc, which aids in the production of thymic hormones, necessary for the functioning of the defense system. The main active ingredients of ginseng are the more than 25 saponin triterpenoid glycosides called “ginsenosides”. These steroid-like ingredients provide the adaptogenic properties that enable ginseng to balance and counter the effects of stress. The glycosides appear to act on the adrenal glands, helping to prevent adrenal hypertrophy and excess corticosteroid production in response to physical, chemical or biological stress. Ginseng is used to restore memory, and enhance concentration and cognitive abilities, which may be impaired by improper blood supply to the brain. Ginseng is also used for diabetes, radiation and chemotherapy protection, colds, chest problems, to aid in sleep, and to stimulate the appetite. In a single daily dosage of the supplement according to the present invention Ginseng is used in an effective amount of 2-8 cc, most preferably 5 cc.

Selenium is a trace mineral; its principal function is to inhibit fat oxidation, often or especially in conjunction with Vitamin E. Selenium protects the immune system, maintains heart, liver, and pancreatic function, and keeps tissues elastic. It protects the liver during cirrhosis from alcoholism. Deficiency is associated with exhaustion, growth impairment, high cholesterol, infection, liver and pancreatic problems, heart disease and cancer. In a single daily dosage of the supplement according to the present invention Selenium is used in an effective amount of 100-300 mg, most preferably 200 mg.

Zinc is essential to the synthesis of DNA and RNA, of proteins, insulin and sperm. The body needs zinc, too, to metabolize carbohydrates, fats, protein and alcohol; to dispose of carbon dioxide; to make good use of vitamin A. More than seventy different enzymes require zinc to do their appointed work. And that's not all. Zinc bolsters the immune system and makes wounds heal faster. It's integral to the growth and maintenance of body tissues; it plays a major role in the development of fetuses and the growth of children. In a single daily dosage of the supplement according to the present invention Zinc is used in an effective amount of 12-38 mg, most preferably 25 mg.

Vitamin B3, Niacin, improves circulation and reduces the cholesterol level in the blood; maintains the nervous system; helps metabolize protein, sugar & fat; reduces high blood pressure; increases energy through proper utilization of food; prevents pellagra; and helps maintain a healthy skin, tongue & digestive system. In a single daily dosage of the supplement according to the present invention Niacin is used in an effective amount of 100-300 mg, most preferably 200 mg.

The above described formulation in the identified effective amounts provides the dietary supplement according to the present invention. The supplement may be provided in daily doses in a liquid carrier, such as a fruit juice (e.g. apple juice or beet juice) or dairy based (e.g. milk or yogurt). The supplement of the present invention may also be formed in daily dosages in a baked good or confection such as a biscuit, cookie, chocolate bar (also known as energy bars). The supplement of the present invention may be in pill or tablet form (with one or more pills/tablets forming a daily dosage. The supplement may also be in powder form with a daily dosage to be added to a carrier liquid, such as water, by the patient. Other forms of the supplement will become apparent to those of ordinary skill in the art. The nutritional supplement according to the preferred embodiment of the present invention contains the most preferred effective amounts of 100 mg vitamin B complex, 1 mg vitamin B₁₂ (Cobalamin), 200 mg Vitamin B₃ (Niacin), 2000 mg vitamin C (Ascorbic acid), 500 mg L-cysteine, 500 mg L-methionine, 200 mg co-enzyme Q₁₀, 10 cc ginko, 1 Tablespoon Mlk Thistle, 5 cc ginseng, 200 mg selenium, 5 cc yerb santa, 5 ml marshmallow root, and 25 mg zinc. The dietary supplement according to the present invention is designed to address the vitamin depletion experienced by users of tobacco products. Further the supplement is designed to cleanse the body of toxins associated with tobacco products and to improve the functioning of the body and the healing of the body. Finally the supplement will assist or prepare the body for the withdrawal of tobacco products, which will assist the user in terminating the use of tobacco products.

What is claimed is:

1. A nutritional supplement comprising effective amounts of vitamin B complex, vitamin B₁₂ (Cobalamin), Vitamin B₃ (Niacin), vitamin C (Ascorbic acid), L-cysteine, L-methionine, co-enzyme Q₁₀, milk thistle, ginko, ginseng, selenium, yerb santa, marshmallow root, and zinc.

2. A dietary supplement comprising effective amounts of vitamins and herbs, wherein the supplement is designed to address the vitamin depletion experienced by users of tobacco products, and the supplement is designed to cleanse the body of toxins associated with tobacco products, to improve the functioning of the body and the healing of the body, and to prepare the body for the withdrawal of tobacco products.

3. The dietary supplement of claim 2 wherein the supplement includes effective amounts of vitamin B complex.

4. The dietary supplement of claim 2 wherein the supplement includes effective amounts of vitamin B₁₂ (Cobalamin).

5. The dietary supplement of claim 2 wherein the supplement includes effective amounts of Vitamin B₃ (Niacin).

6. The dietary supplement of claim 2 wherein the supplement includes effective amounts of vitamin C (Ascorbic acid).

7. The dietary supplement of claim 2 wherein the supplement includes effective amounts of L-cysteine.

8. The dietary supplement of claim 2 wherein the supplement includes effective amounts of L-methionine.

9. The dietary supplement of claim 2 wherein the supplement includes effective amounts of co-enzyme Q₁₀.

10. The dietary supplement of claim 2 wherein the supplement includes effective amounts of milk thistle.

12. The dietary supplement of claim 2 wherein the supplement includes effective amounts of ginko.

13. The dietary supplement of claim 2 wherein the supplement includes effective amounts of ginseng.

14. The dietary supplement of claim 2 wherein the supplement includes effective amounts of selenium.

15. The dietary supplement of claim 2 wherein the supplement includes effective amounts of yerb santa

16. The dietary supplement of claim 2 wherein the supplement includes effective amounts of marshmallow root

17. The dietary supplement of claim 2 wherein the supplement includes effective amounts of zinc.

18. A smoker's nutritional supplement comprising effective amounts of selected vitamins and herbs.

19. The smoker's nutritional supplement of claim 18 comprising effective amounts of L-cysteine, L-methionine, co-enzyme Q₁₀, milk thistle, ginko, ginseng, selenium, yerb santa, marshmallow root, and zinc.

20. The smoker's nutritional supplement of claim 18 comprising 100 mg vitamin B complex, 1 mg vitamin B₁₂ (Cobalamin), 200 mg Vitamin B₃ (Niacin), 2000 mg vitamin C (Ascorbic acid), 500 mg L-cysteine, 500 mg L-methionine, 200 mg co-enzyme Q₁₀, 10 cc ginko, 1 Tablespoon Mlk Thistle, 5 cc ginseng, 200 mg selenium, 5 cc yerb santa, 5 ml marshmallow root, and 25 mg zinc.
